(19) United States
(12) Patent Application Publication
(10) Pub. No.: US 2006/0217698 A1
(43) Pub. Date: Sep. 28, 2006

(54) Ablation of stomach lining to treat obesity

(75) Inventors: Warren L. Starkebaum, Plymouth, MN (US); Thomas R. Prentice, Lake Elmo, MN (US)

Correspondence Address: MEDTRONIC, INC., 710 MEDTRONIC PARK, MINNEAPOLIS, MN, 55432-9924, US

(73) Assignee: Medtronic, Inc., Minneapolis, MN (US)

(21) Appl. No.: 11/389,869

(22) Filed: Mar. 27, 2006

Related U.S. Application Data
(62) Division of application No. 10/424,010, filed on Apr. 25, 2003, now abandoned.

(51) Int. Cl. A61B 18/14

(52) U.S. Cl. 606/32; 606/41

(57) ABSTRACT

The invention provides methods and devices for ablation of stomach tissue to treat obesity. For example, the invention may involve ablation of mucosal tissue to inhibit ghrelin production, recognized as a root cause of increased appetite. The invention alternatively may involve ablation of sub-mucosal tissue to alter myoelectric activity and thereby induce gastroparesis. As a further alternative, gastric muscle tissue, vagal nerves within the stomach or the pyloric region may be ablated to alter stomach function and thereby induce gastroparesis.

### FIELD OF THE INVENTION

The invention relates generally to treatment of obesity and, more particularly, to surgical techniques for treatment of obesity.

### BACKGROUND

A variety of medical approaches have been devised for treatment of obesity, including diet, medication and surgery. Some surgical approaches involve gastric reduction and bypass surgery. U.S. Published Patent Application No. 20020183768 to Deem et al., for example, describes various techniques for reducing the size of the stomach pouch to limit caloric intake as well as to provide an earlier feeling of satiety. Other surgical approaches involve placement of a prosthesis within the stomach of a patient. U.S. Published Patent Application No. 20030040804 to Stack et al., for example, describes a tubular prosthesis that induces feelings of satiety within a patient.

Another surgical technique is described in U.S. Pat. No. 6,427,089 to Knowlton. In particular, Knowlton describes a surgical technique for causing a contraction or reduction in the volume of the stomach by the delivery of thermal energy to the stomach wall. According to Knowlton, the technique relies on a microwave device to heat a submucosal layer of tissue within the stomach wall without thermal damage of the mucosa of the stomach. A resulting thermal lesion causes contraction of the preexisting collagen matrix of the stomach wall.

Another technique for treatment of obesity involves administration of therapeutic agents, such as drugs. For example, extensive research and development has been conducted with respect to appetite suppressants, resulting in limited efficacy and, in many cases, undesirable side effects. Also, PCT Publication No. WO/0187335 to Uhlman et al. describes administration of agents to selectively inhibit ghrelin activity. Ghrelin is a hormone secreted by glands containing parietal cells located principally in the mucosal lining of the stomach. Recent studies suggest that ghrelin is a potent appetite stimulant in animals and man when administered orally. Plasma ghrelin levels have been shown to fluctuate over a 24 hour cycle. In particular, plasma ghrelin levels are elevated before meals, and fall dramatically after meals.

Overweight individuals who lose weight while dieting have increased plasma ghrelin levels compared to levels before weight loss. This observation is consistent with an adaptive homeostatic control mechanism to restore weight to the previous level. In other words, the more weight a patient loses, the stronger the tendency to regain weight. Ghrelin levels have also been shown to be dramatically depressed in patients after gastric reduction and bypass surgery. Presumably, the reduction in ghrelin levels occurs because only a small portion of the stomach, which contains the cells that produce ghrelin, remains intact. The above-referenced Stack et al. document further describes expansion of a prosthesis to contact the walls of the stomach, and inhibit modulation of satiety-controlling factors such as ghrelin.

Table 1 below lists documents that disclose techniques for treatment of obesity.

TABLE 1

| Patent Number | Inventors | Title |
|---|---|---|
| 20020183768 | Deem et al. | Obesity treatment tools and methods |
| 20030040804 | Stack et al. | Satiation Devices and Methods |
| WO/0187335 | Uhlman et al. | Method for selectively inhibiting ghrelin |
| | | action |
| 6,427,089 | Knowlton | Stomach treatment apparatus and |
| | | method |
| 5,782,798 | Rise | Techniques for treating eating disorders |
| | | by brain stimulation and drug infusion |
| WO 00/69376 | Edwards | Surgical weight control device |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY

The present invention is directed to devices and methods for treating obesity. The invention has certain objects. That is, various embodiments of the present invention provide solutions to one or more problems existing in the prior art with respect to treatment of obesity.

The problems include, for example, the ineffectiveness of dieting for many obese patients due to the accompanying increase in ghrelin levels, and the increased appetite that results upon substantial weight loss. Additional problems relate to the general undesirability, invasiveness, infection risk, and recovery time associated with conventional surgical techniques for treatment of obesity, such as gastric reduction and bypass surgery, and other techniques for altering the shape or size of the stomach. Other problems relate to the need for chronic implant of prostheses within the stomach to induce satiety. Further problems include the limited efficacy and side effects of conventional appetite suppressant medications, as well as the uncertain efficacy of medications designed to inhibit ghrelin production, presently in early stages of development, and the need for potential for repeated dosages of such medications by the patient.

Various embodiments of the present invention have the object of solving at least one of the foregoing problems. For example, it is an object of the present invention to overcome at least some of the disadvantages of the foregoing procedures by providing methods and devices for treating obesity that address a root cause of increased appetite, namely ghrelin production. It is an additional object of the invention to provide procedures for inhibition of ghrelin production that are less invasive and present shortened, or even insignificant, recovery times for patients. As a further object, the invention seeks to inhibit ghrelin production with increased efficacy over an extended period of time.

It is another object of the invention to provide methods and devices for treating obesity that involve modulation of stomach function by altering gastric myoelectric activity or muscle function to produce abnormal gastric peristalsis. As examples, objects of the invention include the ability to alter myoelectric activity within regions of the stomach responsible for regulation of myoelectric activity, alter vagal nerve function within the stomach, alter pyloric function, and directly alter gastric muscle function.

Various embodiments of the invention may possess one or more features capable of fulfilling the above objects. In general, the invention provides a method for treating obesity that involves ablating tissue within a stomach of a patient. In some embodiments, the ablation of stomach tissue may be carried out in order to inhibit ghrelin production by the tissue. Ghrelin appears to play a significant role in stimulation of meals and energy balance in humans. The invention provides a way to counteract the effects or ghrelin, or to suppress its secretion from the stomach. In particular, the method may involve ablation of cells that are responsible for production of ghrelin within the mucosal lining of the stomach. Inhibition of ghrelin production suppresses the patient's sensation of appetite.

In other embodiments, the ablation of stomach tissue may be carried out in order to alter gastric myoelectric activity. In particular, the method may involve ablation of cells in various regions of the stomach including the fundus, corpus, and antrum to produce abnormal gastric peristalsis that may be effective in suppressing appetite. The frequency of gastric peristaltic activity is controlled by the so called gastric slow wave, which originates in the pacemaker region of the fundus and propagates slowly toward the antrum, repeating roughly three times per minute. By altering the gastric myoelectric function of the fundus, gastroparesis can be induced to cause slow gastric emptying and loss of appetite in obese patients.

Additional embodiments are directed to the ablation of stomach tissue in the submucosal plexus, myenteric plexus, or both, to destroy cells that regulate myoelectric activity to cause abnormal gastric peristalsis and thereby induce symptoms of gastroparesis. As another alternative, the invention contemplates ablation of gastric muscle to inhibit muscle activity and cause abnormal gastric peristalsis. Abnormal peristalsis by ablation of gastric muscle is expected to result in symptoms of gastroparesis, and thereby cause the patient to lose weight. As further alternatives, the invention provides features relating to ablation of stomach tissue to disrupt the function of the vagal nerve, or ablation of the pylorus, inducing symptoms of gastroparesis and causing weight loss.

Devices for ablation of stomach tissue, either for inhibition of ghrelin production, alteration of gastric myoelectric activity, or alteration of muscle function, may include gastric ablation catheters carrying any of a wide range of ablation probes. In general, the gastric ablation catheter is sized for introduction into the stomach via the esophagus, and carries an ablation probe to provide contact or non-contact ablation of selected regions of the stomach lining.

The ablation probe may take the form of an electrode or array of electrodes for transmission of radio frequency electrical current, optical waveguide for delivery of laser energy, a microwave antenna, a cryogenic probe, an internally heated probe, or the like. In addition, in some embodiments, the ablation catheter may include a fluid delivery port for delivery of fluids to the ablation site for enhanced conductivity or cooling. The ablation level and depth can be controlled to selectively ablated different tissue regions within the stomach and thereby achieve desired effects in treating obesity.

According to one embodiment, the ablation catheter defines a cavity, one or more vacuum ports within the cavity, and an ablation probe that is movable into the cavity. A vacuum source applies vacuum pressure to the vacuum ports to capture a portion of the tissue within the cavity, and the ablation probe ablates at least a portion of the captured mucosal lining.

In another embodiment, the ablation catheter includes an inflatable balloon mounted adjacent a distal end of the ablation catheter, an electrode disposed within the balloon, and a fluid delivery port to deliver fluid to inflate the balloon. The balloon is sufficiently porous to permit flow of the fluid outside of the balloon. A fluid source delivers the fluid to the balloon, and an ablation source delivers electrical current to the tissue via the electrode and the fluid within and outside the balloon. The balloon may be sized so that it can conform to the inner surface of the stomach when inflated, or it may be smaller so that it does not conform. Also, the balloon may have a temperature probe located inside so that ablation can be controlled based on the temperature inside the balloon.

In comparison to known implementations for treatment of obesity, various embodiments of the present invention may provide one or more advantages. For example, the invention avoids the need for invasive, surgical alteration or reconstruction of the stomach, as presented by gastric reduction and bypass procedures, as well as associated patient recovery times. In addition, the invention does not require the implantation of a prosthesis, or administration of medication with uncertain efficacy and prolonged dosage requirements. Rather, the invention provides a surgical ablation treatment that either destroys cells responsible for production of ghrelin, or alters myoelectric activity within the stomach to suppress appetite and thereby treat obesity.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram of a tissue ablation system for use in ablating mucosal stomach tissue to suppress ghrelin production and thereby treat obesity.

FIGS. 2-7 are diagrams of exemplary ablation probes for use with the tissue ablation system of FIG. 1.

FIG. 8 is a diagram of an ablation catheter carrying a radio frequency ablation needle.

FIG. 9 is a side view of an ablation catheter for use in ablating mucosal stomach tissue.

FIG. 10 is a perspective view of a distal end of the ablation catheter of FIG. 8.

FIG. 11 is a side view of an ablation catheter having a curved profile to better conform to a curvature within the stomach.

FIG. 12 is a diagram of a tissue ablation system as shown in FIG. 1, but incorporating a laser ablation probe.

FIG. 13 is a side view of a balloon catheter for use in ablating mucosal stomach tissue.

FIG. 14 is a flow diagram illustrating a method for ablation of stomach tissue to treat obesity.

FIG. 15 is a flow diagram illustrating another method for ablation of stomach tissue to treat obesity.

FIG. 16 is a flow diagram illustrating an additional method for ablation of stomach tissue to treat obesity.

FIG. 17 is a diagram of a tissue ablation system incorporating a thermal balloon for use in ablating mucosal stomach tissue.

FIG. 18 is a diagram of a tissue ablation system incorporating an enlarged thermal balloon for use in ablating mucosal stomach tissue over a larger region of the stomach.

FIG. 19 is a diagram of a tissue ablation system incorporating a thermal balloon sized for ablation of the pylorus.

FIG. 20 is a diagram of a tissue ablation system incorporating a porous balloon for use in ablating mucosal stomach tissue.

FIG. 21 is a diagram of another tissue ablation system incorporating a porous balloon sized for use in ablation of the pylorus.

### DETAILED DESCRIPTION OF THE PREFERRED EMBODIMENTS

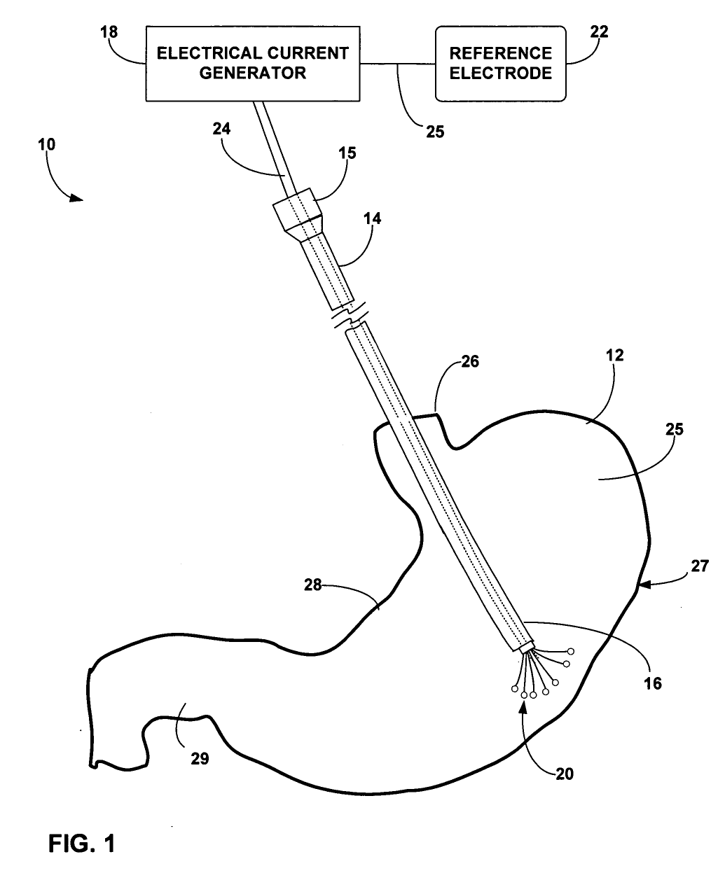

FIG. 1 is a diagram of a tissue ablation system 10 for use in ablating tissue within a stomach 12 to treat obesity. There are multiple targets for ablation within stomach 12, which may be accomplished by appropriate positioning of an ablation probe, and by appropriate selection of ablation parameters. As shown in FIG. 1, system 10 includes a flexible, elongated catheter 14 sized for introduction into stomach 12 via the esophagus (not shown) and antrum 26 of a patient. Catheter 14 includes a proximal end 15 and a distal end 16. In the example of FIG. 1, an electrical current generator 18 delivers electrical current to stomach 12 via an ablation probe 20 and a reference electrode 22. Electrical current generator 18 is coupled to ablation probe 20 via an electrical conductor 24, and to reference electrode 22 via an electrical conductor 25. Reference electrode 22 may be placed on or within the patient, e.g., on the lower back or abdomen.

As will be described, ablation system 10 may incorporate any of a variety of ablation probes, such as an electrode for transmission of radio frequency electrical current, an optical waveguide for delivery of laser energy, a microwave antenna, a cryogenic probe, an internally heated probe, or the like. In addition, in some embodiments, ablation catheter 14 may include a fluid delivery port for delivery of fluids to the ablation site for enhanced conductivity or cooling. The ablation level and depth can be controlled to selectively ablated different tissue regions within the stomach and thereby achieve desired effects in treating obesity. Accordingly, ablation system 10 may be configured and used for a variety of applications involving ablation of stomach tissue.

In accordance with one embodiment of the invention, for example, ablation system 10 may be configured and used to ablate mucosal tissue that lines stomach 12 to destroy cells that produce ghrelin. In this manner, ablation system 10 may be useful in inhibiting ghrelin production and thereby suppressing appetite within the patient to treat obesity. The amount or area of tissue ablated in the mucosal lining of the stomach will determine the level of suppression of ghrelin production, the resulting level of appetite suppression, and hence weight loss by the patient over time.

Ghrelin producing cells are believed to be primarily located in the lining of the stomach, but may be located elsewhere in the gastro-intestinal (GI) tract. Accordingly, ablation system 10 may be applied to ablate ghrelin-producing tissue in stomach 12 and other areas of the GI tract. The energy level and depth of ablation may be selected and controlled by ablation system 10 to selectively ablate the mucosal tissue, and thereby inhibit ghrelin production. The extent of ablation of the mucosa can be measured by measuring the concentration of ghrelin in the blood.

According to another embodiment of the invention, ablation system 18 may be configured and used to ablate sub-mucosal tissue, e.g., in the corpus, fundus, or antrum along either the lesser or greater curvature of the stomach, to alter the myoelectric activity of stomach 12. Hence, the energy level and depth of ablation alternatively may be selected and controlled by ablation system 10 to selectively ablate the submucosal or myenteric plexus, of both, each of which reside at different depths within the stomach lining. In particular, ablation system 10 may be configured to ablate tissue in the submucosal plexus or myenteric plexus to destroy cells that regulate myoelectric activity.

The submucosal plexus is an area just below the mucosa on the upper surface of the gastric muscle, and contains cells that regulate the normal myoelectric activity of the stomach. The myenteric plexus is an area near the outer layers of the gastric muscle that also contains cells that regulate myoelectric activity. The extent of change in the myoelectric activity in the stomach may be accessed by EGG (electrogastrography), using sensing electrodes placed on the skin of the patient's abdomen, embedded into the muscle layers of the stomach, or electrodes placed on the surface of or inserted into the mucosal surface of the stomach.

By altering myoelectric activity in the submucosal plexus, myenteric plexus, or both, ablation system 10 causes abnormal gastric peristalsis, and symptoms of gastroparesis. The initiation and propagation of slow waves occurs within a network of interstitial cells of Cajal, which have the intrinsic ability to spontaneously depolarize three times each minute. These cells are present in the myenteric and submucosal borders of smooth muscle, but have the highest concentration in the pacemaker region in the fundus.

Nausea and vomiting are often associated with abnormal gastric rhythms. For example, tachygastria can be induced by illusory self-motion and occurs prior to the onset of nausea. Gastroparesis is a chronic disorder characterized by abnormally slow gastric emptying and is usually associated with gastric dysrhythmias. Patients with gastroparesis and experience nausea and/or vomiting with meals. As a consequence, these patients often lose weight or become malnourished and may require supplemental nutrition to obtain daily nutritional requirements. However, ablation of stomach tissue to induce gastroparesis can be effective in achieving weight loss for obese, and especially morbidly obese, patients.

As a further embodiment, ablation system 10 may be configured and used to ablate gastric muscle, generally indicated by reference numeral 27, to inhibit muscle activity and cause abnormal gastric peristalsis. Rather than ablate the tissue that regulates myoelectric activity, ablation system 10 ablates the muscle itself to alter muscle function and thereby induce gastroparesis. Abnormal peristalsis by ablation of gastric muscle is expected to result in symptoms of gastroparesis, and thereby cause the patient to lose weight. Again, the energy level and depth of ablation achieved by ablation system 10 can be controlled to selectively ablate the gastric muscle tissue. For assessment of gastric emptying, an ingestible transmitter capsule may be used, as described in U.S. Pat. No. 5,395,366.

In another embodiment, ablation system 10 may be configured and used to ablate submucosal areas containing the vagus nerve. The vagus nerve runs down the esophagus and along the upper aspect of the stomach, and branches multiple times on the outer surface of the stomach. The upper aspect of the stomach refers to the side of the stomach of lesser curvature, generally indicated by reference numeral 28. By ablating the stomach along the lesser curvature with sufficiently high ablation level settings, ablation system 10 can disrupt normal vagal nerve functions, and induce symptoms similar to gastroparesis, including reduced appetite. To assess extent of vagal nerve damage, the extent of heart rate variability can be determined from an ECG or holter monitor.

In an added embodiment, ablation system 10 can be configured and used to ablate the pyloric region, generally indicated by reference numeral 29, to disrupt normal gastric emptying and thereby induce gastroparesis and reduced appetite. For example, ablation of the pylorus may inhibit the function of the pylorus, causing the stomach to empty more slowly. The pylorus can be readily located and accessed with the aid of an endoscope.

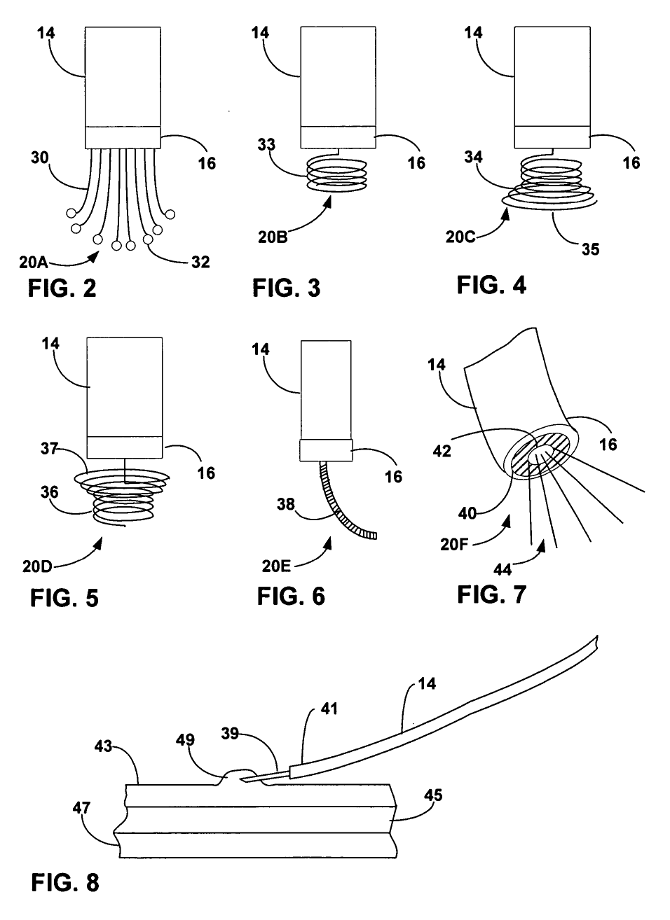

FIGS. 2-7 are diagrams of exemplary ablation probes 20A-20F, respectively, for use with the tissue ablation system of FIG. 1. Ablation catheter 14 and ablation probes 20A-20F may be positioned within stomach 12 using endoscopic imaging techniques or external imaging techniques. For example, an endoscope may be integrated with ablation catheter 14 to facilitate visualization of the area to be ablated, and aid in positioning of ablation probes 20A-20F relative to target tissue within stomach 12.

As shown in FIG. 2, an ablation probe 20A may include a plurality of flexible, electrically conductive filaments 30 that extend from distal end 16 of ablation catheter 14. Each conductive filament 30 is coupled in common to electrical conductor 24 to receive electrical current from electrical current generator 18 (FIG. 1). Conductive filaments 30 may carry spherical electrodes 32. In operation, when distal end 16 is placed in proximity to stomach tissue, flexible filaments 30 extend outward and contact numerous points within a region of tissue to deliver electrical current and thereby ablate the tissue over a larger coverage area. The number of filaments 30 may vary. In addition, filaments may be arranged in a brush-like, two-dimensional array to cover a corresponding area of stomach tissue.

FIG. 3 illustrates an ablation probe 20B comprising a flexible, helical or spiral wound conductive coil 33 that extends outward from distal end 16 of catheter 14. Upon contact with the stomach tissue, coil 33 may compress and expand to more readily conform to a region of the tissue. FIG. 4 illustrates an ablation probe 20C comprising a flexible, helical conductive coil 34 that extends from distal end 16 of catheter 14. In contrast to coil 33 of FIG. 3, coil 34 has an expanded diameter at its distal base 35. FIG. 5 illustrates an ablation probe 20D comprising a flexible, helical conductive coil 36 that extends from distal end 16 of catheter 14. Coil 36 has an expanded diameter at its proximal end 37. FIG. 6 illustrates an ablation probe 20E comprising a flexible, closely wound, conductive coil 38 with a very small diameter relative to coils 32, 34, 36. Each coil 32, 34, 36, 38 contacts stomach tissue and delivers electrical current to ablate the stomach tissue.

FIG. 7 illustrates an ablation probe 20F comprising an electrode 40 and a fluid delivery port 42 at distal end 16 of ablation catheter 14. Fluid delivery port 42 is coupled to a fluid source via a lumen within catheter 14, and delivers a stream of fluid 44. Electrode 40 delivers electrical current to the stomach tissue via fluid 44. Fluid 44 may be electrically conductive, and may be delivered at ordinary body temperatures or cooled temperatures. In this sense, ablation probe 20F may form a virtual electrode, e.g., as described in commonly assigned U.S. Pat. No. 6,537,272 to Christopherson et al., the entire content of which is incorporated herein by reference.

FIG. 8 is a diagram of an ablation catheter 14 carrying a radio frequency ablation needle 39. When distal end 41 of catheter 14 reaches a target tissue site, e.g., a site within mucosal layer 43 adjacent sub-mucosal layer 45, and gastric muscle layer 47, a surgeon inserts needle 39 into the mucosal layer. In the example of FIG. 8, needle 39 is a hollow, conductive needle defining an inner lumen for delivery of fluid. The surgeon delivers an electrolyte fluid, such as saline, into mucosal layer 43 via needle 39 to create a “blister” 49. The surgeon then activates the ablation source, e.g., an electrical current generator, to deliver electrical current to blister 49 via needle 39, and thereby ablate the mucosal region in the vicinity the blister. Alternatively, the surgeon could penetrate deeper into the stomach tissue to form blister 49 within sub-mucosal layer 45 or smooth muscle layers.

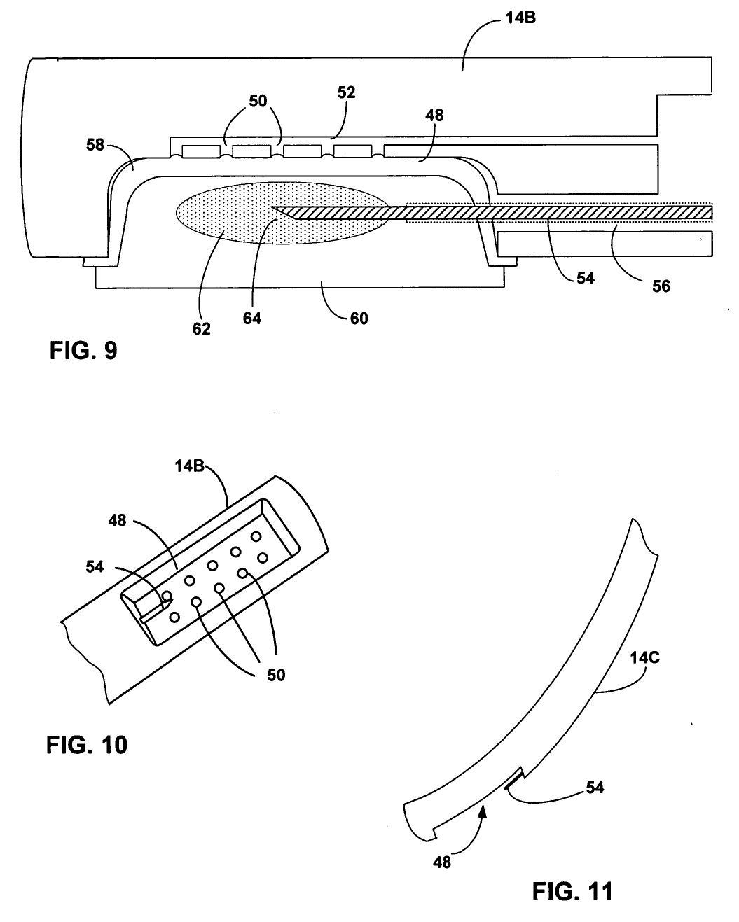

FIG. 9 is a side view of a distal end of an ablation catheter 14B for use in ablating mucosal stomach tissue. FIG. 10 is a perspective view of the distal end of ablation catheter 14B of FIG. 9. As shown in FIGS. 9 and 10, catheter 14B defines a lateral cavity 48 to capture stomach tissue for the ablation procedure. Cavity 48 serves for positioning and fixation of the tissue to be ablated. Cavity 48 includes a plurality of vacuum ports 50 coupled to a vacuum line 52 that extends through catheter 14B. In addition, an ablation probe 54 extends into cavity 48 from a channel 46.

Cavity 48 defines a substantially rectangular orifice or cavity with a major axis extending longitudinally relative to catheter 14B. However, other shapes for cavity 48 are possible. In general, cavity 48 is sized and shaped to permit capture of a selected amount of stomach tissue for ablation. For example, cavity 48 may have different depths for selective ablation of mucosal tissue, sub-mucosal tissue such as submucosal plexus or myenteric plexus, gastric muscle tissue, or tissue containing vagal nerve fibers. In particular, ablation of mucosal tissue may require a relatively shallow cavity depth while ablation of muscle tissue may require an increased depth in order to capture a greater amount of tissue.

Upon deployment of the distal end of catheter 14B to a position within the stomach proximate to a target site, a vacuum source is activated to apply vacuum pressure to vacuum ports 50 to thereby draw the stomach tissue into cavity 48. As an example, a vacuum source may apply an overall negative pressure in the range of 0.01 to 5.0 pounds per square inch (PSI) in order to capture the target tissue. Exemplary dimensions of the cavity 48 are approximately 0.5 mm to 5 mm in width by approximately 1 mm to 10 mm in length. Exemplary cavity depths may be approximately 1 to 4 mm for mucosal ablation, approximately 2 to 6 mm for sub-mucosal ablation, and approximately 4 to 12 mm for gastric muscle ablation. The number and shape of vacuum ports 50, and the pressure applied by each vacuum port, may vary.

While vacuum pressure is maintained, the surgeon extends ablation probe 54 into the captured stomach tissue. The depth of cavity 48, and the height of ablation probe 54 within the cavity, influence the depth of insertion of ablation probe into the captured stomach tissue and hence the precise layer of tissue to be ablated. Ablation probe 54 may be configured to deliver radio frequency electrical current, laser energy, or microwave energy to ablate cells within the captured tissue. In other embodiments, ablation probe 54 may take the form of a cryogenic probe that kills tissue cells by contact or delivery of cryogenic fluid. Alternatively, ablation probe 54 may be internally heated by delivery of electrical current to an internal heating element or closed loop delivery of heated liquid to a probe tip. In this case, ablation probe 54 may ablated tissue by contact heating rather than ohmic heating. As a further alternative, ablation probe 54 may deliver chemical agents to kill or dissolve stomach tissue cells captured within cavity 48.

In the example of FIGS. 8 and 9, ablation probe 54 is a radio frequency conductive needle that carries electrical current delivered by current generator 18 (FIG. 1). The needle may be solid or hollow. In some embodiments, for example, a hollow needle may be used to deliver ablation energy as well as fluid to form a virtual electrode within the tissue or manage cooling of surrounding tissue. In particular, electrical current may be accompanied by delivery of precise volumes of electrolytes to yield desired conduction characteristics.

The electrical current may be selected to provide pulsed or sinusoidal waveforms, cutting waves, or blended waveforms. In addition, the electrical current may include ablation current followed by current sufficient to cauterize any blood vessels that may be compromised during the ablation process. Electrical current flows between ablation probe 54 and a reference electrode placed within or on the surface of the patient's body. Alternatively, in some embodiments, ablation probe 54 may take the form of a bipolar probe that carries two or more electrodes, in which case the current flows between the electrodes.

For ablation of various layers, the electrical current delivers power in the range of approximately 1 to 50 watts, and can be applied at a frequency of approximately 100 to 500 kHz, producing a temperature of approximately 50 to 100 degrees centigrade. To limit ablation of tissue to the target site, the ablation catheter may include multiple temperature sensors for use in closed loop control of the ablation energy so that surrounding tissue can be maintained below 50 degrees centigrade.

In the example of FIG. 9, catheter 14B captures mucosal tissue 58 and sub-mucosal tissue 60 with cavity 48. Ablation probe 54 is positioned to penetrate sub-mucosal tissue 60, and creates a zone 62 of ablated tissue around distal tip 64 of the ablation probe. The size of and volume of zone 62 can be controlled by selection of an appropriate level of electrical current, and may be further controlled by delivery of fluids to form a virtual electrode that extends into interstitial areas and creates a greater overall electrode surface for conduction of ablation energy.

FIG. 11 is a side view of an ablation catheter 14C having a curved profile to better conform to a curvature within the stomach. Ablation catheter 14C may substantially conform to ablation catheter 14B of FIGS. 9 and 10. For example, ablation catheter 14C defines a cavity 48 to capture stomach tissue and includes an ablation probe 54 for ablation of the captured tissue. However, the distal end of ablation catheter 14C has a curved profile selected for better conformance to the shape of the stomach lining.

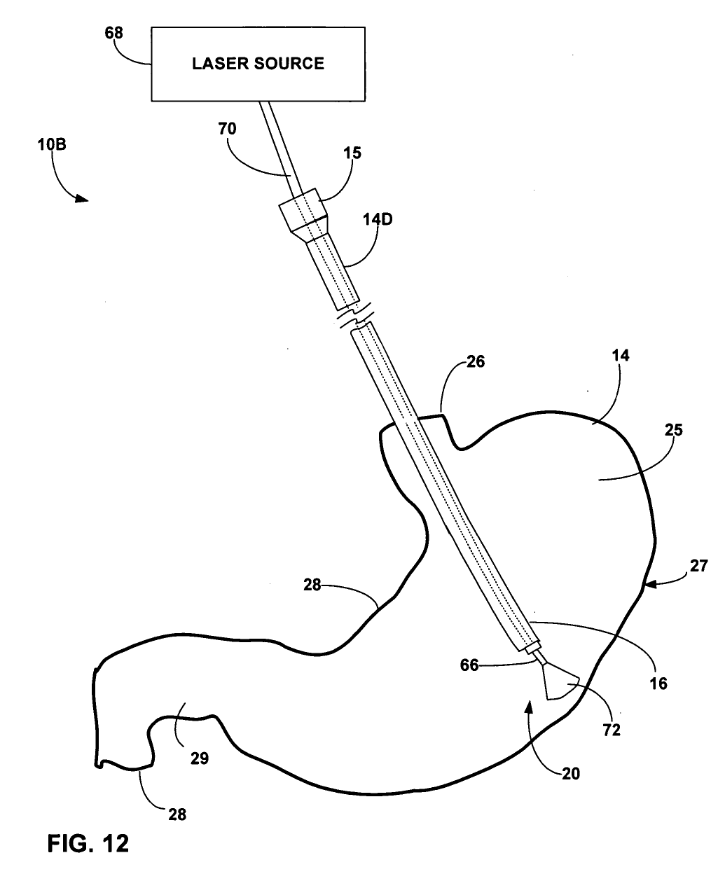

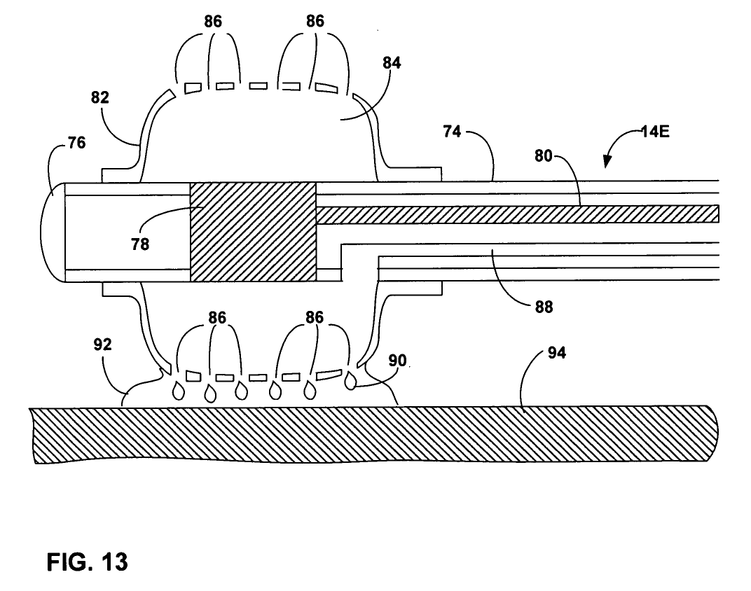

FIG. 12 is a diagram of a tissue ablation system 10B incorporating a laser ablation probe 66. Laser probe 66 receives laser energy from laser source 68 via an optical waveguide 70, such as an optical fiber, and emits the laser energy 72 to ablate tissue within stomach 12. The level and wavelength of laser energy 72 may be selected to target particular tissue sites within stomach 12, such as the mucosal layer for inhibition of ghrelin production, sub-mucosal layers to alter myoelectric activity, gastric muscle to alter muscle function, the vagal nerves, or the pyloric region.

FIG. 13 is a side view of an ablation catheter 14E for use in ablating mucosal stomach tissue. As shown in FIG. 13, ablation catheter 14E includes a flexible catheter body 74 with a distal end cap 76. An electrode 78 is coupled adjacent distal end cap 76 and is coupled to an electrical current source via electrical conductor 80, which extends toward a proximal end of catheter 14E. A flexible, inflatable balloon 82 is mounted about catheter body 74 adjacent electrode 78. Balloon 82 defines an inner chamber 84 and an array of pores 86 sized to permit fluid to leak at a relative low flow rate from the chamber. A fluid channel 88 delivers fluid to chamber 84 of balloon 82 to inflate the balloon. Balloon 82 also has a temperature probe located on an inside of the balloon so the fluid temperature can, if necessary, be precisely controlled.

Upon inflation of balloon 82, fluid droplets 90 are emitted from pores 86, creating a collection of fluid 92 adjacent a tissue site 94. In other words, balloon 82 is inflated with conductive fluid, which is allowed to weep out of pores 86, effectively increasing the surface area of electrode 78. Additionally, the fluid that weeps through balloon 82 serves to distribute heat generated by electrode 78 more evenly across the target tissue. Pores 86 are significantly enlarged in FIG. 13 for purposes of illustration. The mean diameter of pores 86 in the balloon may be in the range of approximately 0.01 to 100 microns.

Application of electrical current to electrode 78 creates a current path between tissue site 94 and a reference electrode (not shown) via the fluid within chamber 84 and the fluid 92 collected at tissue site 94. The fluid within and outside balloon 82 serves to create an virtual extension of electrode 78. Accordingly, the fluid may be an electrolyte selected to provide a desired degree of electrical conductivity. During application of electrical current, fluid channel 88 may continue to deliver fluid to replenish the fluid within chamber 84, and maintain inflation of balloon 82.

The porosity of balloon 82 also makes it possible to measure changes in tissue impedance measured between the electrode 78 in balloon 82 and a reference electrode. This impedance measurement may permit the physician to assess the degree of tissue damage, which is related to the measured impedance. Hence, in addition to visualization using an endoscope or other imaging techniques, catheter 14E may permit the physician to measure electrical impedance as an indication of the level or depth of ablation.

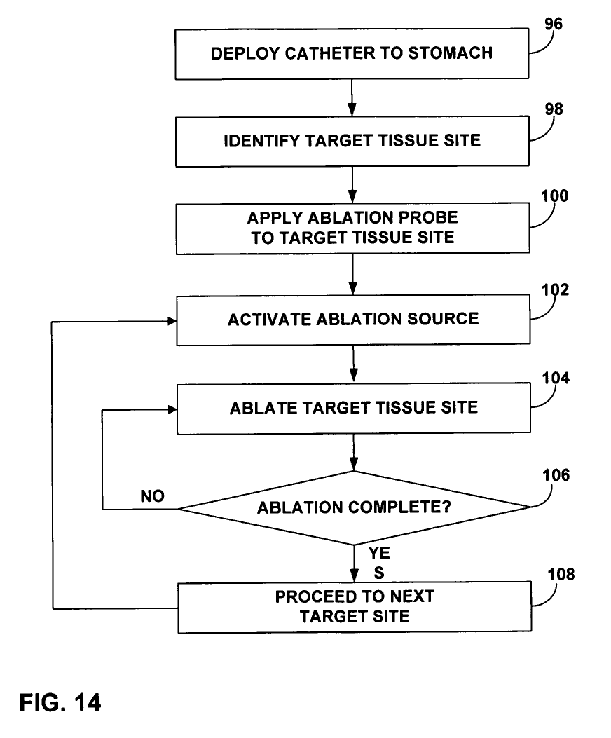

FIG. 14 is a flow diagram illustrating a method for ablation of stomach tissue to treat obesity. The method of FIG. 14 may make use of any of the ablation catheters described herein. As shown in FIG. 14, the method involves deploying an ablation catheter to the stomach (96), identifying a target tissue site within the stomach (98), and applying an ablation probe carried by the ablation catheter to the target tissue site (100). Typically, the patient will be sedated before insertion of the ablation catheter through the patient's mouth and esophagus.

Application of the ablation probe may include placing the ablation probe in contact with or in proximity to the tissue site, depending on the type of ablation probe used. The method further involves activating the ablation source (102) to ablate the target tissue site (104). The ablation source may be activated multiple times until the level ablation is sufficient. When the ablation is complete (106), the method proceeds to the next target site (108). The ablation catheter may be used to ablate multiple tissue target sites, e.g., by moving from site to site during the course of a procedure, to achieve a desired overall effect.

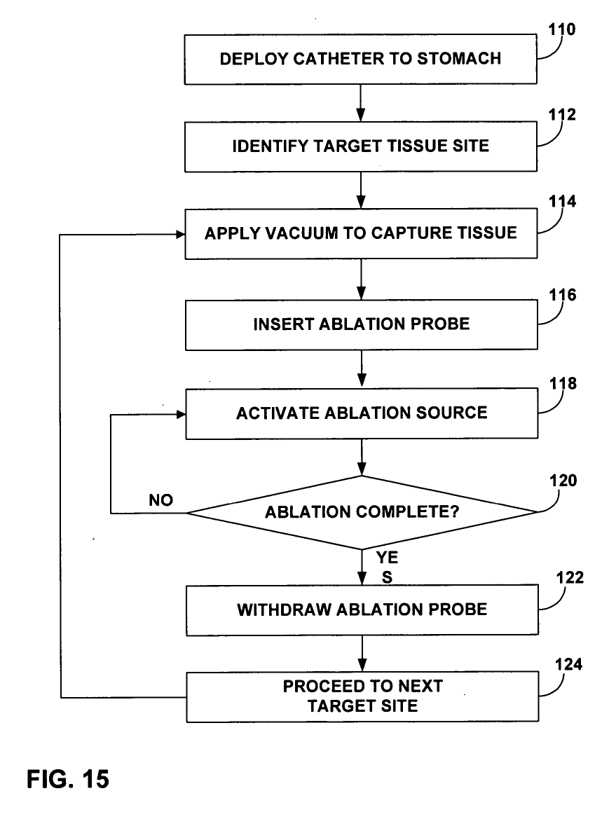

FIG. 15 is a flow diagram illustrating another method for ablation of stomach tissue to treat obesity. The method of FIG. 15 generally involves use of an ablation catheter as illustrated in FIGS. 9-11. In particular, the method involves deploying the catheter to the stomach (110), identifying a target tissue site (112), and then applying vacuum pressure to capture the tissue (114), e.g., in a cavity defined by the catheter. The method then involves insertion of an ablation probe into the captured tissue (116), and activation of the ablation source (118) to ablate the captured tissue. When the ablation is complete (120), the ablation probe is withdrawn from the captured tissue (122), and the method proceeds to the next target tissue site (124).

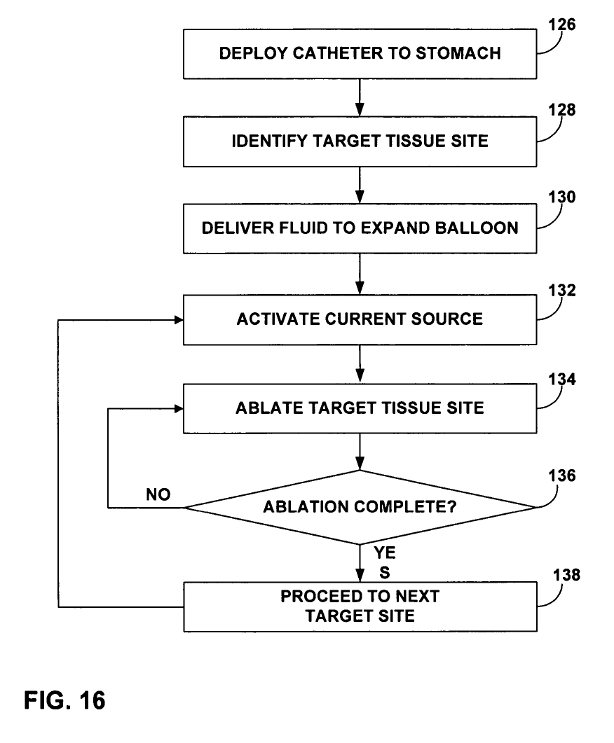

FIG. 16 is a flow diagram illustrating an additional method for ablation of stomach tissue to treat obesity. The method of claim FIG. 15 may make use of a balloon catheter as shown in FIG. 13. As shown in FIG. 16, the method involves deploying the catheter to the stomach (126), identifying a target tissue site (128), and then delivering fluid to inflate a balloon associated with the catheter (130). Upon inflation of the balloon and emission of at least some of the fluid via pores in the balloon surface, a current source is activated (132) to deliver current to an electrode carried within the balloon, and thereby ablate the target tissue (134). When the ablation is complete (136), the method proceeds to the next target tissue site (138). The balloon may remain inflated or be deflated between applications to different target tissue sites.

Before performing an ablation procedure to destroy ghrelin-producing tissue, the physician may measure plasma fasting and pre-meal ghrelin levels in the patient for comparison to post-procedure levels. In the procedure, the surgeon moves the ablation probe between a number of different positions within the stomach until a sufficient amount of tissue in the fundus has been ablated. Over a period of days to weeks after the procedure, a physician again measures plasma fasting and pre-meal ghrelin levels to confirm that the ablation procedure has destroyed enough ghrelin producing cells to significantly lower ghrelin levels circulating within the patient. Ghrelin measurements may be performed using a commercially available assay system such as the ghrelin radioimmunoassay from Linco Research of St. Charles, Mo., or Phoenix Pharmaceuticals of Belmont, Calif.

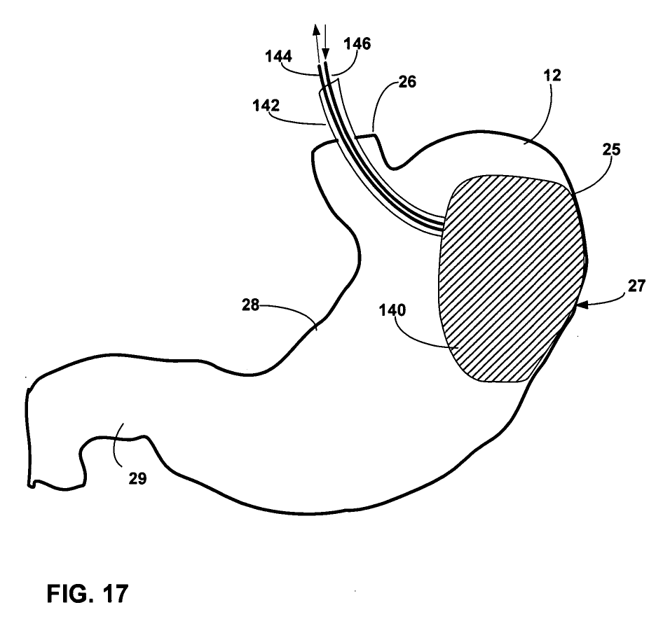

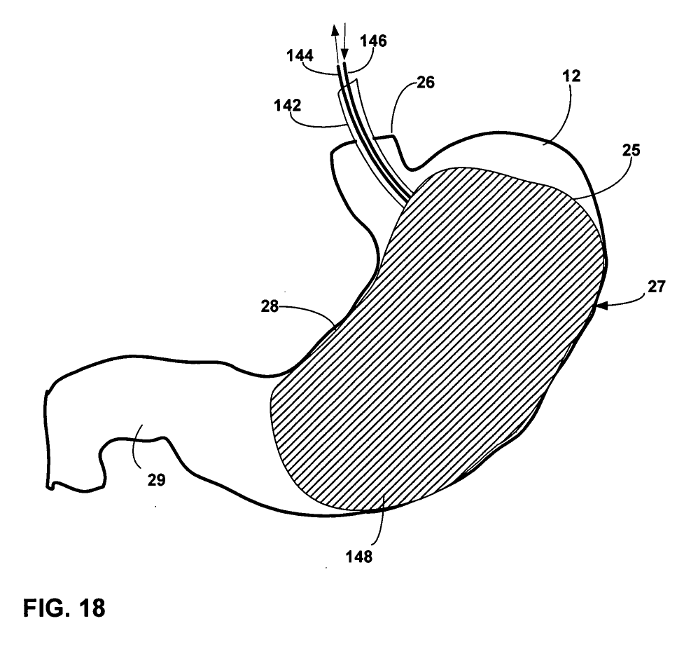

FIG. 17 is a diagram of a tissue ablation system incorporating a thermal balloon 140 for use in ablating mucosal stomach tissue. As shown in FIG. 17, balloon 140 is sized to ablate a substantial area of mucosal tissue within the fundus near the pacemaker region. A conduit 142 coupled to balloon 140 includes a hot fluid return 144 and a hot fluid supply 146 to a fluid delivery device (not shown). Thus, balloon 140 has an inlet and an outlet so that hot fluid can be circulated from an external source of hot fluid. Conduit 142 may be thermally insulated to prevent trauma to the mouth, throat and esophagus of the patient.

In operation, balloon 140 is introduced into the stomach via an endoscope. Fluid, such as water, heated to approximately 50 to 60 degrees centigrade is then circulated with in balloon 140 for a period of time ranging from approximately 10 seconds to 10 minutes, depending on the temperature of the fluid, and the depth of ablation desired. Heat from the hot fluid is transmitted to the stomach through the wall of balloon 140, thereby delivering ablation energy.

In the example of FIG. 17, the stomach can be inflated through a port in the endoscope so that balloon 140 intentionally does not make contact with the entire inner surface area of the stomach in such a way as to avoid the lesser curvature where the vagus nerve is located. Instead, balloon 140 is configured to make contact with the fundus and corpous along the greater curvature of the stomach. This region includes the pacemaker region from which the gastric slow wave initiates and propagates. In some embodiments, balloon 140 may have a more elongate shape to permit contact along a larger extent of the greater curvature of the fundus down to the antrum.

FIG. 18 is a diagram of a tissue ablation system incorporating an enlarged thermal balloon 148 for use in ablating mucosal stomach tissue over a larger region of the stomach. Balloon 148, conduit 142, return 144 and supply 146 generally conform to those illustrated in FIG. 18. However, balloon 148 is sized to contact a larger proportion of the gastric mucosa. In the example of FIG. 18, the stomach may be decompressed via one or the ports in the endoscope (not shown) so that substantially the entire inner surface of the stomach is in contact with balloon 148. The larger balloon 148 and decompression allow for contact, and hence ablation, of tissue along the greater curvature and less curvature from the fundus, corpus, antrum, and also the region of the vagus nerve.

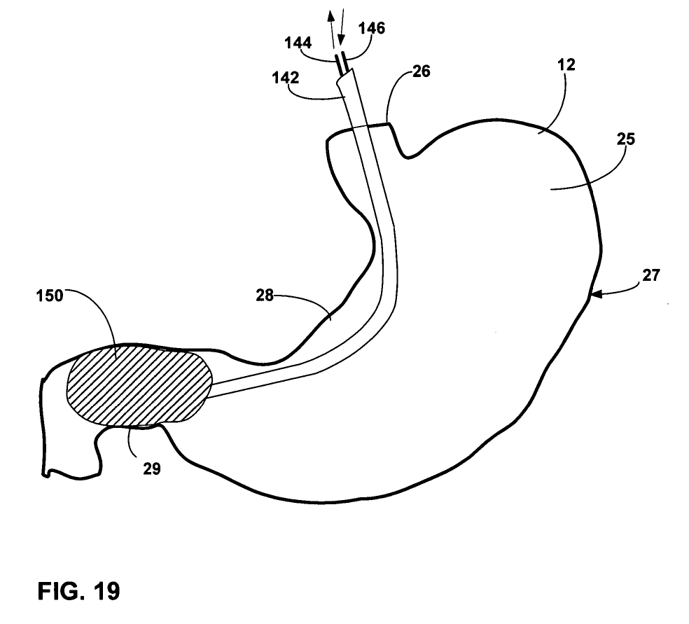

FIG. 19 is a diagram of a tissue ablation system incorporating a thermal balloon 150 sized for ablation of the pylorus 29. As shown in FIG. 19, balloon 150 is coupled to conduit 142, return 144, and 146, as in the examples of FIGS. 17 and 18. However, balloon is sized smaller for positioning within the region of the pylorus 29. Balloon 150 is inserted into the pylorus region 29 and then inflated with hot fluid to ablate the tissue in the region.

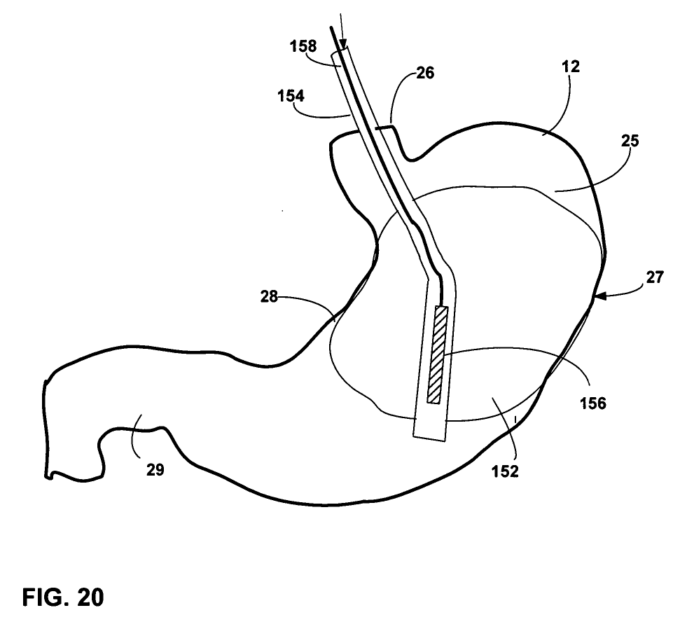

FIG. 20 is a diagram of a tissue ablation system incorporating a porous balloon 152 for use in ablating mucosal stomach tissue. Balloon 152 is mounted about a catheter 154, and includes an array of pores (not shown) to permit gradual emission of fluid from within the balloon to the outside of balloon. Balloon 152 is inflated via an internal lumen within catheter 154. Balloon 152 may be similar to balloon 82 of FIG. 13, but sized larger to better conform to a greater extent of the stomach. As shown in FIG. 20, an RF electrode 156 is mounted within balloon 152 and coupled to an RF current source via a conductor 158. Upon inflation of balloon 152 with electrically conductive fluid delivered via catheter 154, conductor 158 energizes RF electrode 156.

Energization of RF electrode 156 causes transmission of RF current from balloon 152 to the walls of stomach 12 via the conductive fluid emitted through the pores and to a reference electrode mounted on or within the patient, e.g., a ground pad. The progress and extent of tissue destruction occurring during the ablation process may be monitored by observing changes in electrical impedance measured between the RF electrode and the ground pad. In addition, the system can be controlled so that the RF energy is turned off when the temperature measured inside the balloon 152 exceeds a preset temperature limit, such as 55° C., or a preset impedance limit.

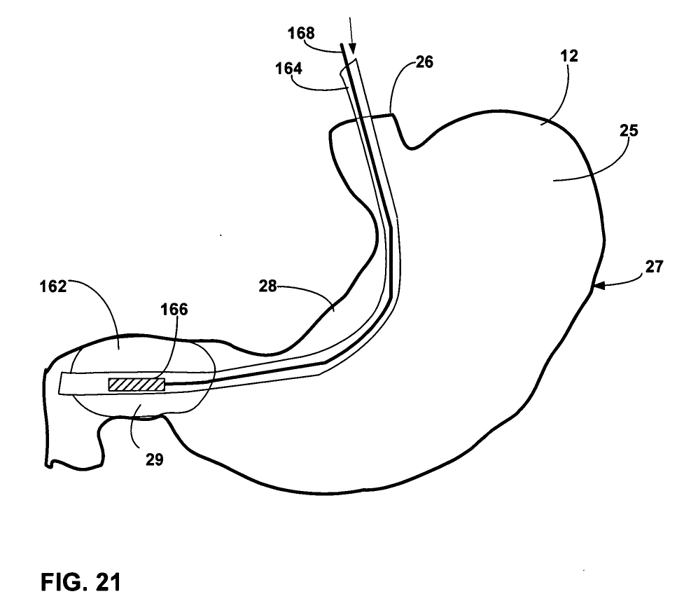

FIG. 21 is a diagram of another tissue ablation system incorporating a porous balloon 162 sized for use in ablation of pylorus region 29. As shown in FIG. 21, porous balloon 162 may be sized for conformance to the inner wall of the stomach within pylorus region 29. Thus, balloon 162 is similar to balloon 152 of FIG. 20, but sized smaller. Like balloon 152, balloon 162 of FIG. 21 is mounted about a catheter 164 and contains an RF electrode 166. RF electrode 166 is coupled to an RF current generator via a conductor 168 within catheter 164. Conductive fluid is delivered to balloon 162 via an internal lumen within catheter 164.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems for ablation, as described herein.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1-17. (canceled)

18. An ablation system comprising:
a catheter sized for introduction into a stomach of a patient and wherein the catheter has a length sufficient to extend into the stomach of the patient via an esophagus of the patient;
an ablation probe disposed at a distal end of the catheter; and
an ablation source to control delivery of ablation energy via the ablation probe in an amount sufficient to ablate tissue within the stomach of the patient and wherein the ablation probe includes one of the group consisting of 1) an array of electrodes suspended at the distal end of the ablation catheter wherein the ablation source is adapted to deliver electrical current to the electrodes; 2) a conductive coil extending from the distal end of the ablation catheter wherein the ablation source is adapted to deliver electrical current to tissue via the electrodes; 3) an optical waveguide extending from the distal end of the ablation catheter, wherein the ablation source delivers laser energy to the tissue via the optical waveguide; 4) a cryogenic probe, wherein the ablation source delivers cryogenic fluid to the cryogenic probe; and 5) a fluid delivery port at the distal end of the ablation catheter and an electrode disposed adjacent the fluid delivery port, the system further comprising a fluid source to deliver fluid via the fluid delivery port and wherein the ablation source is adapted to deliver electrical current to the tissue via the electrode and the fluid; and wherein the ablation source is adapted to ablate tissue at an energy level and depth of ablation chosen to inhibit ghrelin production by the tissue or to alter the gastric myoelectric activity of the tissue.

19. The system of claim 18, wherein the catheter has a length sufficient to extend into the stomach of the patient via an esophagus of the patient.

20-24. (canceled)

25. The system of claim 18, wherein the ablation catheter defines a cavity, one or more vacuum ports within the cavity, and the ablation probe is movable into the cavity, the system further comprising a vacuum source to apply vacuum pressure to the vacuum ports to capture a portion of the tissue within the cavity, wherein the ablation source is designed to deliver ablation energy via the ablation probe to at least a portion of the captured tissue.

26. The system of claim 25, wherein the cavity defines a curvature to conform to a curvature of the stomach.

27. The system of claim 25, wherein the ablation probe defines a conductive needle with a fluid delivery port, the system further comprising a fluid source to deliver fluid to the captured tissue via the fluid delivery port, wherein the ablation source delivers electrical current to the tissue via the electrode and the fluid.

28-44. (canceled)

45. An ablation system comprising:
a catheter sized for introduction into a stomach of a patient wherein the catheter defines a cavity and one or more vacuum ports within the cavity;
an ablation probe disposed at a distal end of the catheter and wherein the ablation probe is movable into the cavity;
a vacuum source to apply vacuum pressure to the vacuum ports to capture a portion of the tissue within the cavity; and
an ablation source to control delivery of ablation energy via the ablation probe in an amount sufficient to ablate tissue within the stomach of the patient to alter gastric myoelectric activity or to inhibit ghrelin production by the tissue.

46. The system of claim 45, wherein the catheter has a length sufficient to extend into the stomach of the patient via an esophagus of the patient.

47. The system of claim 45, wherein the ablation probe includes an array of electrodes suspended at the distal end of the catheter, and wherein the ablation source delivers electrical current to the electrodes.

48. The system of claim 45, wherein the ablation probe includes a conductive coil extending from the distal end of the catheter, and wherein the ablation source delivers electrical current to the tissue via the electrodes.

49. The system of claim 45, wherein the ablation probe includes a fluid delivery port at the distal end of the catheter, and an electrode disposed adjacent the fluid delivery port, the system further comprising a fluid source to deliver fluid via the fluid delivery port, wherein the ablation source delivers electrical current to the tissue via the electrode and the fluid.

50. The system of claim 45, wherein the ablation probe includes an optical waveguide extending from the distal end of the catheter, wherein the ablation source delivers laser energy to the tissue via the optical waveguide.

51-52. (canceled)

53. The system of claim 45, wherein the ablation probe defines a conductive needle with a fluid delivery port, the system further comprising a fluid source to deliver fluid to the captured tissue via the fluid delivery port, wherein the ablation source delivers electrical current to the tissue via the electrode and the fluid.

54. (canceled)
